(19) United States
(12) Patent Application Publication
(10) Pub. No.: US 2010/0215768 A1
(43) Pub. Date: Aug. 26, 2010

(54) NUTRITIONAL SUPPLEMENT

(76) Inventors: Ronald W. Pero, Arlington, VT (US); Stuart Garret, Rancho Mirage, CA (US)

Correspondence Address: KELLY LOWRY & KELLEY, LLP, 6320 CANOGA AVENUE, SUITE 1650, WOODLAND HILLS, CA, 91367, US

(21) Appl. No.: 12/709,890

(22) Filed: Feb. 22, 2010

Related U.S. Application Data
(63) Continuation-in-part of application No. 12/391,431, filed on Feb. 24, 2009.

(51) Int. Cl. A61K 33/30; A61K 31/4406; A61K 31/19; A61K 31/13; A61K 31/047

(52) U.S. Cl. 424/641; 514/356; 514/568; 514/667; 514/729

(57) ABSTRACT

A nutritional supplement includes elements having been shown to enhance DNA repair or reduce DNA damage by different molecular mechanisms while excluding other bioactive nutrient agents which compete with any of the elements known modes of action. The supplement includes a resveratrol, a carotenoid, a nicotinamide, a dimethylaminoethanol, a zinc source, and a quinic acid analog. The supplement is administered daily in dosages in excess of normal dietary levels. The supplement improves resistance to DNA damage, enhances DNA repair capacity in tissues including skin, and stimulates immune cellular function. This was accomplished by reducing metabolic competition between DNA repair bioactive components that was in turn paralleled by enhancement of thirst quenching properties in aqueous formulations.

### BACKGROUND OF THE INVENTION

This invention relates to novel and improved compositions for and methods of treating humans and other animals to reduce DNA damage, enhance DNA repair capacity, and stimulate immune cellular function. More particularly, it relates to the administration to humans (or other animals) a combination of resveratrol, carotenoid, nicotinamide, DMAE and a zinc source material as a drug treatment or a daily dietary supplement, and to compositions containing that combination of materials.

Humans have been selected over hundreds of thousands of years to respond to not just one chemical but to a myriad of chemicals received through the environment and mainly from one's diet. Human physiology is extremely well balanced to handle and process these chemical mixtures to extract as efficiently as possible the necessities of life, such as nutritional energy sources and chemicals to maintain cellular homeostasis and reproduction. This has to be accomplished without introducing any toxicological consequences. Hence, it follows there is a reasonable likelihood that when humans see natural medicines above the levels normally found in the diet or environment, there exists a strong interaction between the megadoses of natural medicines, so that one supplement limits the uptake and metabolism of another, in an effort to provide a natural selection model by which humans can be protected from the toxicological consequences of overdosing. For example, the prior art teaches that carotenoids and vitamins E or C are all radical (electrophilic) scavengers and that these natural products can be combined into supplements for additive biological effects. However, recent literature has not confirmed this practice based on scientific assumption because it was shown that these radical scavengers could inhibit each other's uptake and negate the desired induction of biological effects (Inform 6(7):778-783, 1995; Zhang et al., J. Clin. Nutr. 62:1477 S-1482S, 1995; Niki et al., Am. J. Clin. Nutr. 62:1322S-1326S, 1995).

Selectively administering to humans, as a daily dosage, a combination of carotenoids, nicotinamide (or niacin or a precursor thereof) and a source of zinc, in excess of normal dietary levels, for improving resistance to DNA damage, enhancing DNA repair capacity, and stimulating immune function was disclosed in U.S. Pat. No. 6,020,351, which contents are incorporated herein by reference. The term “carotenoid material” as used herein means carotenoids, such as material selected from the group consisting of alpha carotene, beta carotene, canthaxanthin, lycopene and mixtures thereof. The term “nicotinamide material” as used herein means material selected from the group consisting of nicotinamide, niacin, tryptophan (an amino acid precursor to niacin synthesis) and mixtures thereof. The term Dimethylaminoethanol (DMAE) material, also called deanol, is a naturally occurring substance that has been studied as a possible anti-aging therapy that can also improve cognitive function. The term “zinc source material” as used herein means an appropriate source of zinc for administration to humans and/or other animals, e.g. one or more zinc salts, such as zinc sulfate or other zinc salts like amino acids such as methionine or aspartate, dipeptides, gluconates, halides, nitrates, oxides or acetates. These ingredients have been fully described for use in treating patients having increased DNA damage and decreased DNA repair leading to an impaired immune function in U.S. Pat. No. 6,020,351, and also by Y Sheng, Y, Pero, R W, Olsson, A R., Bryngelsson, C., and Hua, J M entitled “DNA Repair Enhancement by a Combined Supplement of Carotenoids, Nicotinamide, and Zinc” in Cancer Detection and Prevention 1998, 22(4), 284-292.

However, various commercial products so far introduced do not establish or make obvious that any specific combinations of resveratrol, carotenoids, nicotinamide, DMAE and zinc, which are effective at reducing cellular DNA damage induction or enhancing DNA repair and immune function as single agents, can also be additive or synergistic to each other in combination. To the contrary, as demonstrated in U.S. Pat. No. 6,020,351 it was found that the administration of carotenoids, nicotinamide and zinc, when in combination with other natural medicines or nutrients as well, did not reduce cellular DNA damage induction or enhance DNA repair and immune function, as had been assumed but not proven over the years in the prior art. This observation was also consistent with the prior art (Inform 6(7):778-783, 1995; Zhang et al., J. Clin. Nutr. 62:1477 S-1482S, 1995; Nidi et al., Am. J. Clin. Nutr. 62:1322S-1326S, 1995) which has confirmed that carotenoids, nicotinamide and zinc in combination with other natural products (e.g. historic medicines or nutrients) were shown to block each other's uptake and absorption, thus resulting in altered or metabolically blocked biological functions including DNA repair enhancement. It follows then, although not practiced in the prior art, that it cannot be assumed that supplementing a combination of natural products above dietary levels will result in additive biological effects over each product administered separately without previously establishing they possess different modes of action to bring about a common biological effect such as DNA repair enhancement.

Accordingly, there is a need for a combination of ingredients that have been shown to enhance DNA repair and reduce DNA damage by different molecular mechanisms. The present invention fulfills this need and provides other related advantages.

### SUMMARY OF THE INVENTION

The present invention involves the use of a combination supplement consisting essentially of at least two elements having been shown to enhance DNA repair or reduce DNA damage by different molecular mechanisms while excluding other bioactive nutrient agents which compete with any of the elements known modes of action. The elements comprise a resveratrol, a carotenoid, a nicotinamide, a dimethylaminoethanol, a zinc source, and a quinic acid analog. The resveratrol is a polyphenol (e.g. 3, 5, 4′-trihydroxy-stilbene) also known as a phytolexin or phytoestrogen that modulates cell cycle events governing growth arrest. The resveratrol is at least between 30% to 100% pure polyphenol material. The carotenoid is selected from the group consisting of alpha carotene, beta carotene, canthaxanthin, astaxanthin, cryptoxanthin, zeaxanthin, lutein, lycopene and mixtures thereof. The nicotinamide is selected from the group consisting of nicotinamide, niacin, tryptophan and mixtures thereof. The dimehtylaminoethanol (DMAE) is selected from the group consisting of DMAE and choline analogs that cross the blood brain barrier wherein the dimethylaminoethanol contains at least 30% to 100% purity. The zinc source is selected from the group consisting of zinc sulfate, zinc salt amino acids, methionine, aspartate, dipeptides, gluconates, halides, nitrates, oxides and acetates. The quinic acid analog is selected from the group consisting of quinic acid salts, chelates, and Uncaria water extracts.

The resveratrol, carotenoid, nicotinamide, dimehtylaminoethanol, zinc source, and quinic acid analog are all present in proportions above a person's normal daily dosage. A dosage of the resveratrol is about 70-2000 mg per day, the carotenoid is about 100-200 mg per day, the nicotinamide is about 100-200 mg per day, the DMAE is about 100-200 mg per day, the zinc source is about 10-20 mg per day, and the quinic acid analog is about 250-700 mg per day. The supplement may be in a formulation for oral administration or parenteral administration.

An unexpected benefit of combining non-competitive DNA repair enhancers such as resveratrol, carotenoids, nicotinamide/niacin, DMAE and zinc materials into anti-aging products that can synergize enhancement of DNA repair has also been characterized as having powerful thirst quenching properties when administered orally. The Nutra-Reservatrol formulas of DNA enhancers are truly non-competitive in their metabolism, because thirst quenching effects that are non-existent in most single or other nutrient ingredients are in fact synergized to high levels of thirst quenching abilities. Converting individual nutrient ingredients from potentially metabolic competitive mixtures into non-competitive Nutra-Resveratrol formulas are presented herein as biochemical evidence that metabolic competition between Nutra-Resveratrol ingredients does not exist, and thus are in synergized formulations evidenced by enhanced thirst quenching.

Other features and advantages of the present invention will become apparent from the following more detailed description, when taken in conjunction with the accompanying drawings, which illustrate, by way of example, the principles of the invention.

### DETAILED DESCRIPTION OF THE PREFERRED EMBODIMENTS

The present invention is a combination supplement consisting essentially of resveratrol, carotenoid, nicotinamide, DMAE and a zinc source (zinc gluconate) (this combination supplement now called Nutra-Reseratrol) over and above the normal levels of these components in one's diet administered daily, all of which have been shown to enhance DNA repair and reduce DNA damage by different molecular mechanisms, thus permitting a combination of natural products having a non-competitive activation of DNA repair having enhanced potent anti-aging properties. The combination supplement is essentially free of other bioactive nutrient agents that may compete with their known modes of action. The term “bioactive nutrient agents” is employed herein as a generic designation for vitamins, minerals and other bioactive substances serving as anti-oxidants, anti-oxidant co-factors, or otherwise contributing to disease prevention, inhibition of DNA damage, improvement of DNA repair capacity, and/or enhanced immune function, such as have heretofore been sold in concentrated, isolated, or combined form as dietary supplements and the like for human and/or animal consumption. The combination supplement may be embodied in formulations for oral administration, or alternatively, in formulations for parenteral administration.

It was not obvious or practiced in the prior art that resveratrol or DMAE could be combined with other nutritional agents known to enhance DNA repair by avoiding metabolic competition for DNA repair enhancement. For example, even though it has been known since 1998, there have not have been any reports in the literature that resveratrol material also known to be a DNA repair enhancer could be combined with carotenoid material, nicotinamide material, DMAE material, AC-11/quinic acid material or zinc material to create supplements that enhance DNA repair by avoiding metabolic competition for this mode of action. Hence it was not obvious to one skilled in the art that resveratrol could be combined with carotenoids, nicotinamide or zinc salts to enhance DNA repair, because they had different mechanisms of DNA repair enhancement than did resveratrol.

The known molecular mechanisms of resveratrol are described herein. The main effects of resveratrol are to regulate cell cycle events that favor growth arrest allowing DNA repair enhancement before cells die from DNA damage blockage of cell replication (Dario R. Valenzano et al. Current Biology 16 (3): 296-300, 2006; Feng Y H et al. Acta Pharmacol Sin 2002 10: 893-897, 2002; Susanne Andrea Gatz et al. Carcinogenesis 2008 29(3):519-527; Feng Y H et al. Acta Pharmacol Sin 23(10): 893-897, 2002; Lorna Whyte et al. Cancer Res 67(24):12007-17, 2007). There are changes in both gene and protein expression, such as the up-regulation of p53 and p21 and the down-regulation of cyclin A, chk1, CDC27, and Eg5 (a mitotic motor protein). Resveratrol also alters the intracellular Smad signaling of the TGF-β pathway. Finally, dietary restriction, the best-studied life-extension treatment, causes overexpression of SIRT1 (H. Y. Cohen et al. Science 305 pp. 390-392, 2004), and since these effects are not additive to resveratrol, they suggest that a similar molecular mechanism to dietary restriction.

The known molecular mechanisms of carotenoids are described herein. The exact mechanism of action of carotenoids such as beta carotene is not fully understood but it is commonly accepted scientifically that one primary mechanism is to directly scavenge oxygen derived free radicals produced either as by-products of metabolism or from exogenous environmental exposures (Lieber, D. C., Ann. N.Y. Acad. Sci. 691:20-31, 1993; Bohm, F. et al., J. Photochem. Photobiol. 21(2-3):219-221, 1993; Regnault, C. et al., Ann. Pharmacotherapy 27(11):1349-1350, 1993). As a free radical scavenger, carotenoids can be expected to reduce or protect against the chemical damage induced in DNA, RNA and protein of cells by toxic environmental exposures or endogenous cellular metabolic errors that ultimately can result in a disease state. On the other hand, nicotinamide and zinc salts do not possess this chemical property which results in an improved biological cellular function.

The known molecular mechanisms of nicotinamide are described herein. Nicotinamide and its metabolic equivalent nicotinic acid (niacin, vitamin B) or even tryptophan which is the synthetic precursor to niacin is the main precursor for the formation and maintenance of the cellular pool of NAD (Bernofsky, Mol. Cell. Biochem. 33:135-143, 1980; Olsson, A. et al., Biochem. Pharmacol. 45:1191-1200, 1993). NAD is essential for cellular ATP production and maintenance of the cell's redox potential, and it is also the substrate for the DNA repair enzyme, poly ADP-ribosyl transferase (ADPRT). Niacin deprivation decreases the NAD pools significantly both in tissue culture cells (Jacobson, E. et al., IN: ADP-Ribosylation Reactions (Poirier, G. G. and Moreau, P., eds.), pp. 153-162, Springer Verlag, New York, N.Y., 1992), and animal systems (Zhang et al., J. Nutri. 123:1349-1355, 1993) as well as humans (Fu et al., J. Nutri. 119:1949-1955, 1989). The depleted cells have an increased sensitivity to DNA damage and the levels of poly (ADP-ribose) production in cultured cells (Jacobson, E. L., as cited, 1992) or in rat liver (Rawling et al., J. Nutri. 124:1597-1603, 1994) were significantly lower after mild nicotinamide deficiency. On the other hand, when niacin was given as a supplement to ordinary nutrition (i.e. above known dietary levels) the NAD pool increased and the cells were less sensitive to oxygen radicals (Weitberg, A. B., Mutational Res. 216:197-201, 1989). Therefore, it is obvious from this review of the prior art that the primary mechanism of action of nicotinamide/niacin differs from carotenoids and zinc in that the cell's potential for energy metabolism is increased by amplifying NAD and ATP pool supplies (i.e. these biochemicals are the energy sources of living organisms) which in turn is useful to cells, tissues and organs to reduce DNA damage, enhance DNA repair (i.e. poly ADP-ribosylation) and stimulate immune function where the relevance to the disease state is apparent (Pero, R. W. et al., Biochimie 77:385-393, 1995).

The known molecular mechanisms of DMAE material is described herein. Dimethylaminoethanol (DMAE) material, also known as deanol, is a naturally occurring substance that has been studied as a possible anti-ageing therapy that can also improve cognitive function, reduce neurological stress, improves immunity and DNA repair especially in skin. It is the precursor to choline and may increase acetylcholine levels (Grossman R. The role of dimethylaminoethanol in cosmetic dermatology. Am J Clin Dermatol 6:39-47, 2005). While choline is known to be the precursor of acetylcholine, a recognized neurotransmitter, DMAE may also prove to offer a more direct approach to this function by moving into the brain, being acted on by an enzyme (methylation), and thereby undergoing conversion into choline directly where it is needed. DMAE inhibits production of the age-related pigment lipofuscin, which accumulates in all aging tissues. This is significant because cells with increased lipofuscin cause lysosomes to perform poorly, which leads to increased accumulation of poorly functioning mitochondria and increased reactive oxygen species (ROS) production (Terman A, Brunk U T. Oxidative stress, accumulation of biological “garbage,” and aging. Antioxid Redox Signal 8(1-2):197-204, 2006:). Evidence also suggests that DMAE decreases the extent of crosslinking of proteins possibly by acting as a free-radical scavenger (Nagy I, Nagy K. On the role of cross-linking of cellular proteins in aging. Mech Ageing Dev 14(1-2):245-251, 1980).

The known molecular mechanisms of zinc source are described herein. Zinc differs from the resveratrol, carotenoids and nicotinamide with regard to its mechanism of action in that it influences disease development and immune function by being an essential co-factor in several enzyme functions involving replication, DNA repair and antioxidant defense of cells. Zinc is required for cell replication and DNA polymerase activity (Williams, R. O. et al., J. Cell Biol. 58:594-601, 1973). There are two zinc fingers in the DNA binding domain of the poly adenosine diphosphate ribosyl transferase (ADPRT) gene and other DNA repair proteins (Dawat, P. et al., Microbiol. 141 (Pt 2):411-417, 1995; Matsuda, T. et al., J. Biol. Chem. 270(8):4152-4157, 1995; Chiriccolo, M. et al., Mutation Res. 295(3):105-111, 1993) which contain cysteine residues (i.e. an amino acid), and if these cysteine residues are oxidized at their thiol constituents, they would prevent DNA binding and participation in DNA repair (Mazen et al., Nucleic Acid Res. 17:4689-4698, 1989; de Murcia, G. et al., BioEssays 13(9):455-462, 1989; Pero, R. W. et al., Biochimie 77:385-393, 1995; Althaus, F. et al., Mol. Cell. Biochem. 138(1-2):53-59, 1994). Moreover, superoxide dismutase is an antioxidant enzyme protecting cells from the harmful superoxide anion because this radical is a substrate for the enzymatic reaction that also requires zinc as a cofactor (Brunori, M. and Rotilio, G., Methods in Enzymology 105:22-35, 1984).

Resveratrol material is defined herein as a polyphenol (e.g. 3, 5, 4′-trihydroxy-stilbene) that can induce cell cycle arrest thereby permitting greater opportunities to remove DNA damage by DNA repair, and thus stimulate normal cell survival and longevity, which in turn can also result in an increased immune cell function (Dario R. Valenzano et al., Current Biology 16 (3): 296-300, 2006; Feng Y H et al. Acta Pharmacol Sin 2002 10: 893-897, 2002; Susanne and rea Gatz et al. Carcinogenesis 2008 29(3):519-527). These properties for resveratrol materials were not known at the time carotenoid-nicotinamide-zinc materials were described for their non-competitive modes of action in 2000 that directly related to the mechanisms of DNA damage and its repair in U.S. Pat. No. 6,020,151. Thus, this is a novel discovery adding to the previous scientific knowledge already cited.

In a specific aspect, the invention relates to an improved and novel combination of naturally occurring resveratrol material, carotenoid material, nicotinamide material, DMAE material and zinc source material as a combined treatment to aid patients in resisting cellular DNA damage by reducing oxidative cellular damage through enhancing cellular DNA repair capacity (and stimulating immune cellular function) via different modes of action; e.g. cell cycle arrest (resveratrol), oxidative radical scavenger (carotenoids), co-substrate enzymatic stimulator (NAD via nicotinamide), improved mitochrondrial function (DMAE) and a co-factor repair enzyme activator (Poly ADP ribose polymerase, PARP, the DNA binding zinc finger DNA binding region).

In addition, a water extract of Cat's Claw (Uncaria species) called AC-11 or its active ingredient quinic acid analogs are also DNA repair enhancers that do not metabolically compete with resveratrol material, carotenoid material, nicotinamide material, DMAE material or zinc material, and as such could be added to the DNA repair mixture without inducing metabolic competition and thus inhibiting DNA repair instead of being synergist, because the mode of action of AC-11/quinic acid to increasing DNA repair is novel to inducing DNA repair by increasing uptake of urinary tryptophan and nicotinamide (Pero et al Phytotherapy Res 23:335-346, 2009, Pero et al Current Clinical Pharmacology 5: 67-73, 2010).

In another specific sense, these combinations of chemicals known to enhance DNA repair can be used as a dietary supplementation, or as a drug treatment, to prevent (or improve an individual's ability to resist) DNA damage, enhance DNA repair and stimulate immune function in diseases where these processes are central to the manifested disease state; e.g. ageing, cancer, cardiovascular disease and autoimmune disorders such as diabetes, rheumatoid arthritis and ulcerative colitis (Cross et al., Ann. Int. Med. 107:526-545, 1987; Harris, C. C., Cancer Res. 51(Suppl): 5023s-5044s, 1991; Olin, K. L. et al., Proc. Soc. Expt. Biol and Med. 203(4):461-466, 1993).

In summation, even though resveratrol, carotenoids, nicotinamide/niacin, DMAE and zinc have been shown to have some enabling utility in cell and animal models as single agents in the prevention of certain diseases and in the stimulation of immune function and DNA repair, there has been a lack of corresponding, consistent data in humans (Bodgen, J. D. et al., Amer. J. Clin. Nutri. 48:655-663, 1988; Walsh, C. T. et al., Environmental Health Perspectives 102(Suppl. 2):5-46, 1994; Dario R. Valenzano et al. Current Biology 16 (3): 296-300, 2006). In addition, it is not possible for one skilled in the art to predict whether agents with different mechanisms of action will be synergistic or additive to the biological response they will elicit when given in combination. However, the prior art as described and shown in U.S. Pat. No. 6,202,351 does teach that non-competitive molecular mechanisms that enhance DNA repair will at least be additive to each other.

In addition, quite remarkably and an unexpected benefit of combining non-competitive DNA repair enhancers such as resveratrol, carotenoids, nicotinamide/niacin, DMAE and zinc materials into anti-aging products that can synergize enhancement of DNA repair has also been characterized as having powerful thirst quenching properties when administered orally. Since this property has never been observed before for the aforementioned DNA repair enhancers when administered previously as single agents, then this observation becomes unique and proprietary to this patent application. It also supports that the Nutra-Reseratrol formulas (i.e. combinations of resveratrol, carotenoids, nicotinamide/niacin, DMAE and zinc materials) are indeed non-competitive metabolic mixtures, because otherwise they would not synergize their thirst quenching effects in parallel if these effects were also competitive.

The design of the study to prove this invention was based on combining substances with known properties to prevent cancer, stimulate immune function and enhance DNA repair, but with differing mechanisms of action that they did not compete with each other; e.g. combinations of resveratrol=cell cycle modulation, carotenoids=electrophilic scavenger of radicals produced endogenously by cells or exogenously by the environment, nicotinamide=amplified source of energy via increased production of NAD or ATP, DMAE=improved mitochrondial function and zinc=an essential cofactor to antioxidant, replicative and DNA repair enzymes in cells.

The hypothesis was that since none of these DNA repair inducing substances have produced consistent effects in humans as a single administered agent, this shortcoming might be overcome if administered in specific combination, because these substances might produce a consistently additive or synergistic chemo-preventive biological response. This could be true if their mechanisms of action were non-competitive, and thus not inhibiting each other's effects being induced on DNA repair. Here we provide the evidence that Nutra-Reservatrol formulas of DNA repair enhancers are truly non-competitive in their metabolism, because thirst quenching effects that are non-existent in most single or other nutrient ingredients are in fact synergized to high levels of thirst quenching abilities. Converting individual nutrient ingredients from potentially metabolic competitive mixtures into non-competitive Nutra-Resveratrol formulas when in combination are determined and presented here, as biochemical evidence that metabolic competition between Nutra-Resveratrol ingredients does not exist, and thus are in synergized formulations evidenced by enhanced thirst quenching.

In illustrative or preferred practice of the invention, the resveratrol material may be selected from the group consisting of 3, 5, 4′-trihydroxy-stilbene or an equivalent polyphenol in pure or extract form; the carotenoid material may be selected from the group consisting of alpha carotene, beta carotene, canthaxanthin, lycopene and mixtures thereof; the nicotinamide material may be selected from the group consisting of nicotinamide, niacin, tryptophan and mixtures thereof; the DMAE material may be selected from a group consisting of other choline analogs that pass the blood brain barrier and the zinc source material may be one or more zinc salts.

For human administration, the resveratrol material, carotenoid material, nicotinamide material, DMAE material and zinc source material may be present in proportions effective, in combination, to improve resistance to DNA damage, enhance DNA repair capacity, and stimulate immune function in a human subject to whom the composition is administered as a daily dosage.

The invention also contemplates the provision of a method of treating a human or other animal subject, consisting of administering resveratrol material, carotenoid material, nicotinamide material, DMAE material and zinc source material to the subject to selectively supplement the subject's dietary intake thereof (i.e., without supplementing the dietary intake of any other active nutrient agents having competing modes of action) and repeating the administration on a substantially daily basis.

Thus, in a particular sense, the invention contemplates the provision of a method of treating a human subject consisting of selectively administering to the subject resveratrol, carotenoid, nicotinamide, DMAE and a zinc source in daily dosage amounts effective, in combination, to improve resistance to DNA damage, enhance DNA repair capacity, and stimulate immune function. In a specific example of currently preferred dosage range for humans, about 100-2000 mg resveratrol, about 100 mg of carotenoid, about 100 mg of nicotinamide, about 100 mg DMAE, and about 10 mg of a zinc source are administered daily in this method.

From a theoretical standpoint, this invention is based on a principle of combining chemical products which are individually known to possess either cancer preventive or immune stimulatory properties into one formulation which contains only active components where at least one mechanism of action for each active component is known to be different from the known mechanisms of action of the other components to stimulate DNA repair. So far as applicant is aware, this particular combination of ingredients has not heretofore been recognized in the art.

The invention involves the discovery that natural products should not be combined into a natural medicine unless one tests whether each ingredient is additive to the overall desired biological effect, and that one way to accomplish this endpoint is to not combine natural products that have similar modes of action and thus competitive routes of absorption and excretion without first testing the combination for additive effects. That is to say, the present invention avoids inhibited uptake and absorption of natural products, thereby obtaining additive biological effects, by combining only natural products having well defined different and thus potentially non-competitive modes of action which is, for example, the case with the exclusive combination of resveratrol, carotenoids, nicotinamide, DMAE, and zinc.

The practice of this invention involves supplementing humans or animals for example, by the oral, intraperitoneal, intravenous, subcutaneous or intramuscular routes of administration with the combination of resveratrol, carotenoids, nicotinamide/niacin, DMAE and an appropriate zinc salt at a dose of this combination that exceeds a normal dietary supplementation. The practice of the prior art teaches that dietary supplementation containing this combination together with simultaneous supplementation of other nutrients and/or natural products cannot enhance immune function (Payette, H. et al., Am. J. Clin. Nutr. 52:927-932, 1990; Zhang et al., J. Clin. Nutr. 62:1477 S-1482S, 1995; U.S. Pat. No. 6,020,351) but when resveratrol, carotenoids (as Caroplex, C.E. Jamieson, Ltd., Ontario, Canada), nicotinamide, DMAE and a zinc salt are given alone in the absence of other natural supplements above dietary levels, e.g. 70-2000 mg per day, 100 mg per day, 100 mg per day, 100 mg per day and 10 mg per day by oral daily administration, respectively, the resistance to oxidative cellular DNA damage, and enhancement of DNA repair and immune function were observed.

The clinical evaluation in a previous study (U.S. Pat. No. 6,020,351) was determined by comparing each individual's biological response before and after supplementation. In such a manner, each individual became his own control; e.g. the male subjects were given baseline measurements of resistance to cellular DNA damage, enhancement of DNA repair and stimulation of immune function once a week for 4 weeks, and then they were supplemented daily and the same measurements repeated once a week for the last 5 weeks of a 7 week intervention period. The before measurements (i.e. n=4) were the baseline biological response parameters to be compared to the after measurements (i.e. n=5). One individual was not supplemented to provide a control for the supplemented individuals. The data from this experimental design has taught that resistance to cellular DNA damage, enhancement of DNA repair and stimulation of immune function were all significantly modulated by a combination of carotenoids, nicotinamide, and zinc when administered as an exclusive drug combination above dietary levels, but not when co-administered together with other additional nutrient or natural product supplements.

The following examples (Tables 1-5) are some preferred features but not limitations to this invention. The compositions of DNA repair enhancers presented herein contain both well-known bioactive DNA repair components as well as inert ingredients pertinent to beverage formulations rendering the products favorable for smell, taste, color, viscosity and water solubility. Whereas the so-called inert ingredients do not necessarily vary from one formulation to another, it is sometimes highly desirable to vary the bioactive ingredients of formulations to achieve high specificity and duration of biological responses. As already mentioned, the DNA repair ingredients may vary in amounts (doses) from one formulation to another in Tables 1-5, e.g. resveratrol=0.07 gm to 2.0 gm per 1000 gm water, carotenoids=0.01 gm to 0.3 gm per 1000 gm water, nicotinamide=0.01 gm to 0.3 gm per 1000 gm water, DMAE=0.01 gm to 0.3 gm per 1000 gm water, or Zinc=0.001 gm to 0.03 gm per 1000 gm water.

TABLE 1
Nutra-Resveratrol-Lemon Lime

| | GRAMS |
|---|---|
| WATER | 986.358 |
| SODIUM BENZOATE | 0.33 |
| YELLOW 5 - COLOR | 0.002 |
| BLUE 1 -COLOR | 0.00004 |
| CAFFEINE | 0.135 |
| REBIANA 09201 CARGILL STEVIA | 0.19 |
| FRUCTOSE | 9.4 |
| NIACINAMIDE | 0.012 |
| ZINC GLUCONATE | 0.001 |
| DMAE | 0.012 |
| RESVERATROL | 0.07 |
| SF LEMON LIME FLAVOR EXT | 1.2 |
| CLOUD EMULSION SF | 0.6 |
| ESTER GUM-STABILIZER | 0.05 |
| GUM ARABIC-EMULSIFIER | 0.14 |
| CITRIC ACID | 1.5 |
| | 1000 |

TABLE 2
Nutra-Resveratrol Orange Mango (FD 4227.3)

| | GRAMS |
|---|---|
| WATER | 986.228 |
| POTASSIUM SORBATE | 0.33 |
| BETA CAROTENE 10% - color | 0.05 |
| REBIANA 09201 CARGILL STEVIA | 0.19 |
| FRUCTOSE | 9.4 |
| CANTAXANTHIN | 0.012 |
| NIACINAMIDE | 0.012 |
| ZINC GLUCONATE | 0.001 |
| DMAE | 0.012 |
| RESVERATROL | 0.07 |
| SF ORANGE FLAVOR EXT | 1.2 |
| SALT | 0.03 |
| SODIUM CITRATE | 0.025 |
| MONO POTASSIUM PHOSPHATE | 0.09 |
| ASCORBIC ACID | 0.06 |
| CLOUD EMULSION SF | 0.6 |
| ESTER GUM-STABILIZER | 0.05 |
| GUM ARABIC-EMULSIFIER | 0.14 |
| CITRIC ACID | 1.5 |
| | 1000 |

TABLE 3
Nutra-Resveratrol-Pomegranate Berry (FD 4227.2)

| | GRAMS |
|---|---|
| WATER | 982.788 |
| SODIUM BENZOATE | 0.33 |
| CARAMEL COLOR DS | 0.025 |
| SENSIENT GRAPE RED-color | 1.87 |
| REBIANA 09201 CARGILL STEVIA | 0.19 |
| FRUCTOSE | 9.4 |
| CANTAXANTHIN | 0.012 |
| NIACINAMIDE | 0.012 |
| ZINC GLUCONATE | 0.001 |
| DMAE | 0.012 |
| RESVERATROL | 0.07 |
| SF RASPBERRY WONF FLAVOR | 2.5 |
| SF POMEGRANATE FLAVOR | 0.5 |
| CLOUD EMULSION SF | 0.6 |
| ESTER GUM-STABILIZER | 0.05 |
| GUM ARABIC-EMULSIFIER | 0.14 |
| CITRIC ACID | 1.5 |
| | 1000 |

TABLE 4
Nutra-Reservatrol-Kiwi Strawberry) (FD 4227.4)

| | GRAMS |
|---|---|
| WATER | 984.983 |
| SODIUM BENZOATE | 0.33 |
| ANTHO RED -(vegetable and fruit color) | 0.8 |
| REBIANA 09201 CARGILL STEVIA | 0.19 |
| FRUCTOSE | 9.4 |
| CANTAXANTHIN | 0.012 |
| NIACINAMIDE | 0.012 |
| ZINC GLUCONATE | 0.001 |
| DMAE | 0.012 |
| RESVERATROL | 0.07 |
| SF STRAWBERRY FLAVOR | 0.4 |
| SF WATERMELON FLAVOR | 1.5 |
| CLOUD EMULSION SF | 0.6 |
| ESTER GUM-STABILIZER | 0.05 |
| GUM ARABIC-EMULSIFIER | 0.14 |
| CITRIC ACID | 1.5 |
| | 1000 |

TABLE 5
Nutra-Resveratrol-Citrus Drink (FD 4227-5)

| | GRAMS |
|---|---|
| WATER | 987.54 |
| SODIUM BENZOATE | 0.375 |
| REBIANA 09201 CARGILL STEVIA | 0.19 |
| FRUCTOSE | 9.4 |
| NIACINAMIDE | 0.012 |
| ZINC GLUCONATE | 0.001 |
| DMAE | 0.012 |
| SF LEMON LIME FLAVOR EXT | 1.2 |
| RESVERATROL | 0.07 |
| CITRIC ACID | 1.2 |
| | 1000 |

The total sample size for each evaluation was 3 fluid ounces (about 90 ml): i.e. after this consumption there is no more can be supplied. Each subject consumes a 1 2×2 inch saline cracker (Nabisco Original Saltine Crackers) before tasting each new product identified as products #A (Minute Maid Lemonade), #B (Nutra-Resveratrol Lemon Lime), #C (Gatorade), #D (Nutra-Resveratrol Orange Mango), or #E (Poland Spring Water). After the saline and first tasting the self-report clinical examination form is filled out for ranking of thirst quenching only (Table 2), and in addition as influenced by taste, appearance and smell of the products (Table 3) according to the scale provided of 1=worst ranking, 2, 3, 4, 5=best ranking. The subject is instructed to first rank all products for thirst quenching, and then repeat the process also taking into consideration taste, smell and appearance of the products. Once the subject has decided sample ranking the data are then compared to water (#E). Ranked Values greater than water (#E) are considered to have an improved thirst quenching ability over water alone.

Below is a sample summary data sheet for all self-reported clinical examinations used for final analysis.

TABLE 6
Example of the self-report clinical evaluation form

| | |
|---|---|
| Patient Name: | Birth date: |
| Treatment: | |

| | Initial Ranking of Thirst | Also Taste, |
|---|---|---|
| | Quenching Effects Including | Appearance, |
| | Duration of Response for | Smell |
| Observations | Products #A, #B, #C, #D | Ranking |
| Body Weight (kg) | | |
| Height | | |
| BMI | | |
| #A | Thirst quench Value | Rank = |
| Treatment | #1*, #2**, #3***, #4****, #5***** | |
| #B | Thirst quench Value | Rank = |
| Treatment | #1*, #2**, #3***, #4****, #5***** | |
| #C | Thirst quench Value | Rank = |
| Treatment | #1*, #2**, #3***, #4****, #5***** | |
| #D | Thirst quenchValue | Rank = |
| Treatment | #1*, #2**, #3***, #4****, #5***** | |
| #E | Thirst quench Value | Rank = |
| Treatment | #1*, #2**, #3***, #4****, #5***** | |
| | #1* = worst | 1 = worst |
| | #2**, #3***, #4**** | 2, 3, 4 |
| | #5***** = best | 5 = best |

Below are the results of ranking of thirst quenching only for the following products according to self report clinical examination forms: Minute Maid Lemonade, Nutra-Reservatrol lemon lime, Gatorade, Nutra-Resveratrol orange mango, Poland Spring Water.

TABLE 7
Thirst Quench Product Identity

| | | Lemonade | Nutra-R. | | Nutra-R | Water |
|---|---|---|---|---|---|---|
| Subject | | Minute | lemon | | orange | Poland |
| Identity | Age | Maid | lime | Gatorade | mango | Spring |
| 1. MP | 61 | 3 | 4 | 2 | 3 | 1 |
| 2. IP | 21 | 1 | 4 | 3 | 3 | 2 |
| 3. EP | 17 | 3 | 3 | 3 | 1 | 3 |
| 4. GA | 53 | 4 | 3 | 3 | 2 | 5 |
| 5. HA | 15 | 4 | 4 | 4 | 5 | 1 |
| 6. CH | 18 | 2 | 5 | 4 | 3 | 1 |
| 7. NH | 17 | 2 | 1 | 5 | 5 | 4 |
| 8. OH | 13 | 3 | 5 | 3 | 4 | 5 |
| 9. NWH | 49 | 5 | 3 | 3 | 5 | 1 |
| 10. JH | 46 | 4 | 1 | 3 | 5 | 2 |
| 11. JW | 69 | 5 | 4 | 2 | 2 | 1 |
| 12. VO | 66 | 5 | 5 | 3 | 3 | 5 |
| 13. MO | 69 | 5 | 3 | 3 | 1 | 1 |
| 14. BA | 55 | 5 | 4 | 3 | 3 | 2 |
| 15. RP | 70 | 3 | 5 | 3 | 4 | 3 |
| Total raw score | | 54 | 54 | 47 | 49 | 37 |
| % Improvement | | 45.9% | 44.1% | 27.0% | 32.4% | 0 |
| compared to water | | | | | | |

n = 14

Evaluation of thirst quenching together with taste, smell, and appearance by comparison of the following products according to the self report clinical examination forms: Minute Maid Lemonade, Nutra-Reservatrol lemon lime, Gatorade, Nutra-Resveratrol orange mango, Polan Spring water. The data sheets for self-report clinical examinations used here, equally rank thirst quenching only from 1-5, and together with taste, appearance and smell from 1-5, and then report the sum of these two assessments as the final score herein. The composition of Nutra-Resveratrol lemon lime or orange mango flavors can be found in Table 1. Lemonade Minute Maid, Gatorade and Water Poland Spring are commercial products where their ingredients are defined publicly on every can or bottle sold. As such they are among the best examples of thirst quenchers already being sold to the public.

TABLE 8
Thirst Quench + taste, appearance and smell of product

| | | Lemonade | Nutra-R. | | Nutra-R | Water |
|---|---|---|---|---|---|---|
| Subject | | Minute | lemon | | orange | Poland |
| Identity | Age | Maid | lime | Gatorade | mango | Spring |
| 1. MP | 61 | 6 | 8 | 5 | 5 | 2 |
| 2. IP | 21 | 2 | 8 | 5 | 6 | 5 |
| 3. EP | 17 | 7 | 6 | 7 | 2 | 7 |
| 4. GA | 53 | 9 | 6 | 7 | 4 | 9 |
| 5. HA | 15 | 9 | 7 | 8 | 7 | 2 |
| 6. CH | 18 | 6 | 8 | 9 | 5 | 2 |
| 7. NH | 17 | 5 | 2 | 9 | 8 | 7 |
| 8. OH | 13 | 8 | 10 | 7 | 6 | 7 |
| 9. NWH | 49 | 10 | 5 | 5 | 8 | 2 |
| 10. JH | 46 | 9 | 2 | 5 | 10 | 5 |
| 11. JW | 69 | 9 | 9 | 7 | 6 | 2 |
| 12. VO | 66 | 9 | 10 | 5 | 5 | 7 |
| 13. MO | 69 | 10 | 6 | 6 | 2 | 3 |
| 14. BA | 55 | 10 | 9 | 6 | 6 | 5 |
| 15. RP | 70 | 6 | 10 | 6 | 7 | 3 |
| Total raw score | | 115 | 106 | 97 | 88 | 68 |
| % improvement | | +69.1% | +55.9% | +42.7% | +29.4.1% | 0 |
| compared to water | | | | | | |

n = 15

In conclusion, Tables 7 and 8 have presented data demonstrating that Nutra-Resveratrol ingredients (Tables 1-5) which are characterized as DNA repair mixtures of resveratrol, carotenoid, nicotinamide, DMAE, and zinc gluconate that do not metabolically compete with each other because they modulate DNA repair by different molecular mechanisms that in turn achieve an additive or synergistic effect. Biochemical evidence is presented in Tables 7 and 8 because individual Nutra-Resveratrol ingredients have never been shown to have thirst quenching effects. However, when they are combined in non-competitive mixtures, there is also parallel metabolic synergy of other biochemical properties such as enhanced thirst quenching) (Tables 7-8).

Moreover, when Nutra-Resveratrol formulas or other commercial thirst quenchers, such as lemonade or Gatorade, were directly compared to water they all were more effective, scoring between 32.4% to 45.9% higher than water as shown on Table 7. This was true even in taste, appearance, and smell were also taken into consideration being 29.4% to 69.1% better than water (Table 8).

All the thirst quenching formulas were positively influenced (i.e. increased) by their formulations when compared to water except Nutra-Resveratrol orange mango which did not increase its thirst quenching value (Tables 2-3). Apparently Nutra-Resveratrol orange mango was not a preferred formulation mixture when considering taste, appearance and smell for these DNA repairs.

The role of calories and sugars in DNA repair synergized formulas with thirst quenching properties compared to commercial beverages and water was also looked at. It is striking how different Gatorade and lemonade are from Nutra-Resveratrol formulas in regard to calories and sugar content (see Table 9 below).

First of all, Nutra-Resveratrol mixtures were formulated as anti-aging beverages having enhanced DNA repair health benefits, whereas Gatorade and lemonade only have dehydration and thirst quenching health benefits. Reducing calories from 50-100 per serving as it is with Gatorade and lemonade to 10 per serving as it is in Nutra-Resveratrol formulas is in itself a large health benefit, since weight gain is a potent inducer of bad health. To achieve this added benefit, and yet still have both anti-aging and thirst quenching properties as in the Nutra-Resveratrol formulas, has to be considered a very favorable development and not predicted by the scientific prior art.

All the thirst quenching formulas tested also had sugars present in their formulas (Table 9). The commercial products (Gatorade and lemonade) had much higher levels of 14-27 gm per serving compared to 2 gm per serving for the Nutra-Resveratrol formulas tested. Sugars in general are also an unhealthy source to stimulate any beneficial nutritional metabolism. Although sugars may enhance thirst quenching properties as evidenced by the data presented in Tables 7-8, they are questionable ingredients adding no further health benefit beyond preventing dehydration.

TABLE 9
The level of calories and sugars in beverages demonstrating
thirst quenching properties compared to water.

| | Per Serving of 8 Fluid Ounces | |
|---|---|---|
| Name of Beverage | Calories | Sugars (total carb. gm) |
| Nutra-Resveratrol Lemon lime | 10 | 2 |
| Nutra-Resveratrol Orange mango | 10 | 2 |
| Gatorade | 50 | 14 |
| Minute Maid Lemonade | 100 | 27 |
| Poland Spring Water | 0 | 0 |

Although several embodiments have been described in detail for purposes of illustration, various modifications may be made to each without departing from the scope and spirit of the invention. Accordingly, the invention is not to be limited, except as by the appended claims.

What is claimed is:

1. A nutritional supplement, comprising:
at least two elements having been shown to enhance DNA repair or reduce DNA damage by different molecular mechanisms while excluding other bioactive nutrient agents which compete with any of the elements known modes of action, wherein the at least two elements are chosen from the group consisting of a resveratrol, a carotenoid, a nicotinamide, a dimethylaminoethanol, a zinc source, and quinic acid analog.

2. The supplement of claim 1, consisting essentially of a resveratrol, a carotenoid, a nicotinamide, a dimethylaminoethanol, and a zinc source wherein the supplement is essentially free of other bioactive ingredients.

3. The supplement of claim 1, consisting essentially of a resveratrol, a carotenoid, a nicotinamide, a dimethylaminoethanol, a zinc source, and quinic acid analog wherein the supplement is essentially free of other bioactive ingredients.

4. The supplement of claim 1, 2 or 3, wherein the resveratrol is a polyphenol (e.g. 3, 5, 4′-trihydroxy-stilbene) also known as a phytolexin or phytoestrogen that modulates cell cycle events governing growth arrest.

5. The supplement of claim 4, wherein the resveratrol is at least between 30% to 100% pure polyphenol material.

6. The supplement of claim 1, 2 or 3, wherein the carotenoid is selected from the group consisting of alpha carotene, beta carotene, canthaxanthin, astaxanthin, cryptoxanthin, zeaxanthin, lutein, lycopene, and mixtures thereof.

7. The supplement of claim 1, 2 or 3, wherein the nicotinamide is selected from the group consisting of nicotinamide, niacin, tryptophan and mixtures thereof.

8. The supplement of claim 1, 2 or 3, wherein the dimehtylaminoethanol (DMAE) is selected from the group consisting of DMAE and choline analogs that cross the blood brain barrier.

9. The supplement of claim 8, wherein the dimethylaminoethanol contains at least 30% to 100% purity.

10. The supplement of claim 1, 2 or 3, wherein the zinc source is selected from the group consisting of zinc sulfate, zinc salt amino acids, methionine, aspartate, dipeptides, gluconates, halides, nitrates, oxides and acetates.

11. The supplement of claim 1 or 3, wherein the quinic acid analog is selected from the group consisting of quinic acid salts, chelates, and Uncaria water extracts.

12. The supplement of claim 1, 2, or 3, wherein the resveratrol is a polyphenol (e.g. 3, 5, 4′-trihydroxy-stilbene) also known as a phytolexin or phytoestrogen that modulates cell cycle events governing growth arrest and the resveratrol is at least between 30% to 100% pure polyphenol material, and wherein the carotenoid is selected from the group consisting of alpha carotene, beta carotene, canthaxanthin, astaxanthin, cryptoxanthin, zeaxanthin, lutein, lycopene, and mixtures thereof, and wherein the nicotinamide is selected from the group consisting of nicotinamide, niacin, tryptophan and mixtures thereof, and wherein the dimehtylaminoethanol (DMAE) is selected from the group consisting of DMAE and choline analogs that cross the blood brain barrier and the dimethylaminoethanol material contains at least 30% to 100% purity, wherein the zinc source is selected from the group consisting of zinc sulfate, zinc salt amino acids, methionine, aspartate, dipeptides, gluconates, halides, nitrates, oxides and acetates, and wherein the quinic acid analog is selected from the group consisting of quinic acid salts, chelates, and Uncaria water extracts.

13. The supplement of claim 12, wherein the resveratrol, carotenoid, nicotinamide, dimehtylaminoethanol, zinc source, and quinic acid analogs are all present in proportions above a person's normal daily dosage.

14. The supplement of claim 13, wherein a dosage of the resveratrol is about 70-2000 mg per day.

15. The supplement of claim 13, wherein a dosage of the cartenoid is about 100-200 mg per day.

16. The supplement of claim 13, wherein a dosage of the nicotinamide is about 100-200 mg per day.

17. The supplement of claim 13, wherein a dosage of the DMAE is about 100-200 mg per day.

18. The supplement of claim 13, wherein a dosage of the zinc source is about 10-20 mg per day.

19. The supplement of claim 13, wherein a dosage of quinic acid analog is about 250-700 mg per day.

20. The supplement of claim 13, wherein a dosage of the resveratrol is about 70-2000 mg per day, wherein a dosage of the cartenoid is about 100-200 mg per day, wherein a dosage of the nicotinamide is about 100-200 mg per day, wherein a dosage of the DMAE is about 100-200 mg per day, wherein a dosage of the zinc source is about 10-20 mg per day, and wherein a dosage of quinic acid analog is about 250-700 mg per day.

21. The supplement of claim 20, wherein the supplement is in a formulation for oral administration.

22. The supplement of claim 20, wherein the supplement is in a formulation for parenteral administration.
